(19) United States
(12) Patent Application Publication
(10) Pub. No.: US 2008/0243394 A1
(43) Pub. Date: Oct. 2, 2008

(54) SYSTEM, METHOD AND COMPUTER PROGRAM PRODUCT FOR MANIPULATING THERANOSTIC ASSAYS

(75) Inventors: Emanuel F. Petricoin, Gainesville, VA (US); Lance A. Liotta, Bethesda, MD (US)

Correspondence Address: VENABLE LLP, P.O. BOX 34385, WASHINGTON, DC, 20043-9998, US

(73) Assignee: Theranostics LLC, Rockville, MD (US)

(21) Appl. No.: 12/057,163

(22) Filed: Mar. 27, 2008

Related U.S. Application Data
(60) Provisional application No. 60/907,288, filed on Mar. 27, 2007.

(51) Int. Cl. G01N 33/569; C12Q 1/02; G06F 19/00

(52) U.S. Cl. 702/19; 435/29; 435/7.21

(57) ABSTRACT

A theranostics technique for describing signaling pathway activity within a cellular or tissue sample may include analyzing a cellular sample to obtain sample quantitative values for a series of target protein modification levels reflected in a set of a plurality of protein biomarkers in the sample. The sample quantitative values may be compared to reference quantitative values for the same series of protein modification levels. The reference quantitative values may be statistically processed from a plurality of comparable samples. The sample quantitative values may be displayed in relation to the reference quantitative values in a way that may suggest a specific course of treatment.

### RELATED APPLICATIONS

The present application is a non-provisional application claiming priority to U.S. Provisional Application No. 60/907,288, filed Mar. 27, 2007, the contents of which are incorporated herein in their entirety.

### FIELD OF THE INVENTION

The present invention is related to theranostic assays. More specifically, the invention relates to systems, methods and computer program products for manipulating and displaying the results of theranostic assays.

### BACKGROUND OF THE INVENTION

Many new cancer therapies have been developed, but patient outcome has not changed much over the past several decades. In 2005, approximately 1.4 million new cases of cancer were diagnosed in the U.S. Per year, about 559,650 Americans are expected to die of cancer, more than 1,500 people each day. Many treatments are unsuccessful. Therapy is very costly. Most therapeutic success rates are about 20 to 30 percent.

It is difficult to predict which patient will respond to which therapy, and there is an urgent need to predict which patients will respond to a given therapy so that each patient gets the right therapy. Often, therapies do not work in all patients and cancer remission is frequently temporary. Therapies may cause toxic, debilitating side effects in many patients, without benefit, and they are expensive.

The term “theranostics” combines therapy and diagnostics, and is used generally to describe the use of diagnostic testing to diagnose a disease, choose the correct treatment regime, and monitor the patient response to therapy. Theranostics may provide improved healthcare through better disease management.

Theranostic tests have not yet been fully accepted, either as laboratory-based-tests or point-of-care (POC) tests, despite advances in proteomics, genomics and pharmacogenomics. Alliances between diagnostic and genomics companies have not met the need for predictive medicine and disease management in theranostics.

Theranostics can dramatically improve the efficiency of drug treatment by helping physicians identify patients who are the best candidates for the treatment in question. In addition, the adoption of theranostics could eliminate the unnecessary treatment of patients for whom therapy is not appropriate, resulting in significant drug cost savings for these patients.

Most drugs, especially for oncology, target protein functions. There is an unmet need to measure the activity of the actual protein drug targets in order to specifically tailor therapies for various diseases.

For example, cancer can be understood as a disease of the cellular signal network brought about by genetic alterations that result in hyperactive protein pathways that drive growth. Mutations may activate or inactivate key proteins, thereby driving cancer. As a consequence, the altered protein circuit gives the cancer a survival advantage over cells with protein activity in normal ranges. Thus, there is a need to measure the state of activity of the actual drug targets (e.g. the proteins) in a patient's individual cancer.

Significant barriers still remain before theranostics can be broadly applied to medical treatment for cancer and other diseases. Some of these obstacles include selecting the appropriate assays, processing the resulting data, presenting it to physicians, insurers, patients, and others in a useful format, storing the data consistent with regulatory and privacy requirements, and retrieving the data for patient follow up. Despite these barriers, there is a great need for companies developing diagnostic tests to predict susceptibility to certain conditions, to benefit participants in the health care system.

### SUMMARY OF THE INVENTION

Embodiments of the present invention are directed to systems, methods and computer program products to address the problems described above.

The reporting method provides a useful means for processing, distilling, communicating and visualizing important clinical and theranostic information for a physician or other user in a simple form suitable for medical or scientific decision making.

According to one embodiment, the invention may comprise a reporting system for describing the quantity and activity state of molecules (analytes) within a cellular or tissue sample. It may start with a selected panel of defined molecular endpoints within the cellular signaling network listed in a tabular form. Each of these endpoints may describe a molecule that is involved in cellular signaling/signal transduction or components of cellular metabolic pathways. Each one of these endpoints, in turn, may either be a drug target or directly associated with a drug target or linked to a drug target by residing within the molecular pathway of a drug target. For example, selected analytes could provide a portrait of the activity level of pathways involving those involving cell growth, cell death, survival, differentiation, stress, etc.

Within the report, the activity level of each analyte may be provided in a numerical or qualitative description in comparison to a reference standard so that the analytes that fall out of the normal range may be highlighted. For example, the list of selected analytes could be provided in columnar form, and the levels of each analyte displayed.

In another embodiment, the tabular report may be supplemented by a diagram that resembles a street map with a highlighted route. These highlighted routes may refer to the pathway that is activated insofar as representing the drug targets, the activity values of which are above the normal range. It may be a cellular interconnected network comprising one or more of the reported analytes that are members of the network. These highlighted routes, thus, form an easy-to-understand representation of a new or well known cellular network or pathway that is in an active state based on the activity level of the analyte or analytes reported.

In further embodiments, the report may be used for assisting a therapeutic decision, and supplemented by a listing of drugs known to those skilled in the art to target one or more components of the highlighted route or pathway on the map diagram, or one or more of the analytes listed as outside the normal range in the report.

In one example embodiment, the analyte activity level may be a quantitative value of the phosphorylation state of the analyte compared to a reference value. Such a reference value could be the level of that phosphorylation in a population of control samples, the level of phosphorylation in a cell line treated with a ligand or a phosphatase inhibitor, or the level of phosphorylation in a purified sample of the analyte of known concentration.

According to yet another embodiment, the invention may comprise a method for making a therapeutic decision comprising the steps of: (a) analyzing a cellular sample to obtain a quantitative value for a series of activity levels reflected in a specific and predefined number of protein post translational modifications; (b) reporting the activity level in a tabular form, and or diagrammatic form, to highlight those analytes falling out of a reference standard range; and (c) selecting a therapy or therapies from a list of drugs that act on targets associated with the highlighted analyte.

Alternatively, the method may comprise a method for making a therapeutic decision wherein step (a) above may be modified. The specified and predetermined number may be the least number of endpoints that comprise “nodes” within a broader cellular network, culled from a much broader number of possible endpoints. One embodiment may comprise a computer algorithm-defined minimal number of measurements at “nodes” within the cellular signaling or metabolic pathway “circuit” that allows the broadest possible measurement of a network. These nodes may be chosen based on key intersection points within the network that define and comprise those derangements known within human disease subcategories, such as (a) human cancer without regard to type of cancer; (b) diabetes; (c) cardiovascular disease; (d) inflammation; (e) infectious disease; (f) ocular diseases (e.g., macular degeneration); and (g) neurodegenerative diseases (e.g., Alzheimer's disease).

All of the above embodiments may be implemented in multiple forms, e.g., as an apparatus, as a method, as hardware, as firmware, and as a computer program product in the form of software on a computer-readable medium. Regarding the latter, the invention may be embodied in the form of a computer system running such software. Furthermore, the invention may be embodied in the form of an embedded hardware device running such software.

In one embodiment, the invention may be a computer-implemented method of manipulating theranostic assays, comprising: analyzing a sample of cells to obtain a series of sample quantitative values of protein modification levels for a set of target proteins, the set being sufficient to define one or more signaling pathways; comparing the sample quantitative values to reference quantitative values of protein modification levels for the same set of target proteins, wherein the reference quantitative values are statistically processed from a plurality of comparable samples; and displaying the sample quantitative values in relation to the reference quantitative values.

In another embodiment, the invention may be a theranostics system, comprising: a computer network, including server means and a plurality of clients in communication with said server means over said network; means for operating said server means and said plurality of clients, said operating means supporting a run-time environment for a theranostics application on said network; graphical user interface means adapted to be displayed on said plurality of clients; a database storing a plurality of files with a plurality of different file formats; a plurality of collaborative modules, each of which is adapted to be run over said network, said collaborative modules including: an assay planner to determine which target proteins to assay; and a data analyzer to analyze a sample of cells to obtain sample quantitative values for a series of target protein modification levels reflected in a set of a plurality of target proteins in the sample and to compare the sample quantitative values to reference quantitative values.

In another embodiment, the invention may be a method of operating a theranostics system, comprising: obtaining patient data from a client computer; analyzing the patient data on a server to obtain sample quantitative values for a series of target protein modification levels reflected in a set of a plurality of target proteins in the sample; comparing the sample quantitative values to reference quantitative values for the same series of target proteins wherein the reference quantitative values are statistically processed from a plurality of comparable samples; and displaying the sample quantitative values in relation to the reference quantitative values on the client computer.

In another embodiment, the invention may be method of characterizing a disease in a subject, comprising: selecting a set of target proteins of a plurality of signaling pathways, wherein the signaling pathways involve modifications of the target proteins, obtaining a set of quantitative reference values for a reference range of modification levels for each of the target proteins in the set, wherein a modification level outside the reference range for one or more of the target proteins indicates a deranged signaling pathway in the cell associated with a disease, measuring modification levels for the set of target proteins in cells of a patient, determining, for each target protein in the set, whether the modification level is within or outside the reference range, and displaying the measured modification levels of the set of target proteins in the sample and the reference range of modification levels for the target proteins.

In another embodiment, the invention may be a method of classification and characterization of a disease state in a cell, comprising: selecting a set of target proteins of a plurality of cell signaling pathways, wherein the signaling pathways involve modifications to the target proteins, obtaining a set of quantitative values for a reference range of modification levels for each of the target proteins in the set, wherein the level in one or more of the target proteins indicates a relative state of activation in a signaling pathway in the cell associated with a disease as compared to modifications in target proteins within the reference population of patient values, measuring modification levels for the set of target proteins in cells of a patient, determining, for each target protein in the set, whether the modification level is high or low compared to the reference population of patients, and displaying the modification levels of the set of target proteins in the sample and the reference range of modification levels for the target proteins.

### BRIEF DESCRIPTION OF THE DRAWINGS

Specific embodiments of the invention will now be described in further detail in conjunction with the attached drawings, in which:

FIG. 1 depicts a simplified block diagram of the systems according to embodiments of the present invention;

FIG. 2 depicts a block diagram of an embodiment of a computer that may be used to implement embodiments of the present invention;

FIG. 3 shows an example of a diagram report for two patients, according to an embodiment of the invention, using signaling pathway highlights with FIG. 3A showing activation of an Akt signaling pathway, and FIG. 3B showing activation of a MAPK signaling pathway;

FIG. 4 shows an embodiment of the invention in the form of a data handling flow chart, from collection of the patient samples to data transmission to the doctor;

FIG. 5 shows an example of a set of biomarkers with normalized activity levels collected from a set of patients;

FIG. 6 shows an example of a signaling network diagram, with FIG. 6A showing activation of an Atk signaling pathway, and FIG. 6B showing activation of a MAPK signaling pathway;

FIG. 7 shows an example of a variant of a Drug Target Activity Report;

FIG. 8 shows another example of a variant of a Drug Target Activity Report;

FIG. 9 shows an example of a line graph plot as an example of a data display;

FIG. 10 shows an example of a box plot as an example of a data display;

FIG. 11 shows an example of a bihistogram as an example of a data display;

FIG. 12 shows an example of a standard deviation plot as an example of a data display;

FIG. 13 shows an example of a mean plot as an example of a data display

FIG. 14 shows an example of a star plot as an example of a data display, with 14A patient data per protein, and 14B showing protein data per patient; and

FIG. 15 shows an example of a radar plot as an example of a data display.

### DETAILED DESCRIPTION

### Definitions

The following definitions are applicable throughout this disclosure, including in the above.

A “computer” may refer to any apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

A “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

“Software” may refer to prescribed rules to operate a computer. Examples of software include: software; code segments; instructions; computer programs; and programmed logic.

A “computer system” may refer to a system having a computer, where the computer comprises a computer-readable medium embodying software to operate the computer.

“Proteomics” may refer to the study of the expression, structure, and function of proteins within cells, including the way they work and interact with each other, providing different information than genomic analysis of gene expression.

“Theranostic assays” as used herein refers to a wide array of assays, including those that are generally considered to be diagnostic of disease in a traditional sense, those that are used to determine an appropriate therapy for a disease, assays that are used to monitor therapy, and fully theranostic assays that combine features of two or more of diagnosis, therapy selection, and therapy monitoring.

The invention relates to signaling pathways, otherwise referred to as cell signaling pathways, signal transduction pathways, or signal cascades. Such pathways may involve intracellular protein modifications induced by an external signal, such as the binding of a ligand to a receptor at the cell surface. The receptor may be an enzyme that modifies itself and/or another protein in response to binding to a ligand, and transduces, or passes, the signal to the next protein in the pathway, or cascade. This process allows cells to communicate with their environment, and to pass the messages within the cell, to produce particular molecular biological results. Pathway activation may also result from genetic mutations which confer constitutive activation (e.g., phosphorylation) to a protein analyte based on changes in protein folding and protein-protein interactions, or mutations that result in loss of negative regulators (e.g. mutations in the PTEN phosphatase cause constitutive activation of AKT protein in cells_).

Various types of protein modifications may be involved in signaling. For example a kinase receptor phosphorylates proteins, and phosphorylation may produce a binding site for a different protein, inducing a protein-protein interaction with the next protein downstream. For example, MAPK signal transduction pathways are named after mitogen-activated protein kinase (MAPK), which phosphorylates downstream target proteins and ultimately can alter gene transcription and cell division.

Signaling pathways may be complex multi-component systems with a variety of cell-surface receptor triggers, and various intracellular target proteins providing intracellular feedback and signal amplification. Moreover, there may be many interactions between target proteins causing or being modified in response to multiple signals from multiple signaling pathways. For a protein that is characterized as being part of a unique signaling pathway, modification of that protein, e.g. phosphorylation, indicates activity of that signaling pathway. However, many proteins are involved in two or more signaling pathways. Detecting modification of such proteins may be insufficient to identify activation of a particular unique signaling pathway. However, if two or more proteins of the pathway are modified, that may be sufficient to identify activation of a unique signaling pathway.

According to the invention, a practitioner may review available literature regarding signaling pathways and the proteins within them, and may identify and select a minimum number of proteins that are necessary to define a particular unique signaling pathway from the broader cellular network of interconnected signaling pathways and the very large number of proteins involved within them. Antibodies are available for many modified forms of proteins of signaling pathways. The final set of target proteins selected according to the invention may include target proteins with modified forms for which antibodies are available, wherein modification of the target proteins reflects activity of their signaling pathway(s). More specifically, modification of a particular group of the target proteins may indicate which unique pathway or pathways are active.

Further, the activity of particular pathways, and the modification of proteins within them, may be characterized as within a range of activation within any given affected population cohort (e.g. breast cancer patients). Relative activation levels of any given patient within the cohort can be classified and characterized in relation to the entire population distribution and categorized as high and low (or average) as compared to the rest of the patient population values. Moreover, many drugs are known to specifically modulate and act on the proteins of the signaling pathways, and thereby modulate activity of the pathway (increasing or decreasing activity, acting as agonists or antagonists/inhibitors). The select set of target proteins may be those that are known to be targets of particular available drugs.

Thus a novel aspect of the invention is that any given patient value is directly compared to protein activation levels from other similarly affected patients in order to determine if the protein of interest is activated and “in use”. For example, analysis of phosphorylated c-Kit protein from a biopsy specimen obtained from a patient with breast cancer is compared to the population distribution of c-kit phoshporylation from other breast cancer patients' tissue samples. A simple embodiment of the analysis is to simply ascertain if the new patient value is within the top or bottom quartile of the population distribution and report a “high” or “low” classification/categorization based on that value. Moreover, the new patient value is then added to the population data and the data bank value becomes an adaptive evolutionary database which can become more accurate over time when combined with clinical outcome data. This analytical means is based on the growing knowledge that each patient's disease such as cancer is based on a constellation of patient-specific mutational events

Thus, the inventive methods provide a surprisingly effective tool for health care providers. The invention involves displaying information sufficient to determine which proteins within a given patient's cells have levels of protein activity that are high or low compared to the population distribution based on protein activity values obtained from patients of a comparable and similar disease cohort. This information may also include which pathways are active in cells of the patient, and may further include a list of the drug or drugs that may be likely to have a desirable therapeutic effect for that patient, by modulating the protein modification levels, and the signaling pathway activity, from a high value to a low value. The doctor or other health care practitioner can thus readily determine which of the available therapies are appropriate for the particular patient. The samples of cells used in the inventive assay methods may be from a patient or from a cell culture. The target proteins in the cells are typically analyzed in their extracted form, for example by reverse phase arrays, or they may be analyzed without destroying the cells in various types of immunohistology, flow cytometry and other techniques.

Embodiments of the invention may involve measuring and reporting the activity of actual drug targets (e.g. proteins) in a patient biopsy. The inventive system may involve identifying key protein drug targets' activity changes in human cancer or other diseases, optimizing technologies to measure the activity in the protein drug targets in human biopsies, and correlating the activity with therapeutic responses. The biopsy method may involve collecting as few as 1000 cells, and the assay may involve measuring hundreds of proteins at once using protein microarrays.

An embodiment of the invention may establish a new type of proteomics report. A proteomics report may be a panel or suite of information that can be reported to physicians to improve therapy decisions for their patients. With such a report, cancer and other diseases with a common diagnosis may be stratified at a molecular level, according to the therapies that are likely to be effective.

In the research context, embodiments of the invention may provide a method for drug screening and reporting of drug effects on cell lines with extension into preclinical and clinical trials. The inventive methods can be used to identify new drug targets, assess the effectiveness of anticancer drugs and other therapeutic agents, improve the quality and reduce costs of clinical trials, discover the subset of positive responders to a particular drug (stratifying patient populations), improve therapeutic success rates, and/or reduce sample sizes, trial duration and costs of clinical trials.

In the health care context, embodiments of the invention may provide a service to physicians that will enable the physicians to tailor optimal personalized patient therapies. For example, a tissue specimen may be sent by the pathologist and/or clinical oncologist to a theranostics laboratory facility, e.g. one operated by Theranostics Health, LLC. The laboratory may analyze the tumor's cell circuitry to identify the “renegade pathways” that are causing the cancer to grow unimpeded. The laboratory may provide the treating pathologist or clinical oncologist with a report listing the activated drug targets in the patient's tumor. The report may give the physician a new class of information about the patient's individual cancer or other disease. This may enable physicians to tailor therapy to the individual patient's tumor or other disorder, prescribe the right therapy to the right patient at right time, provide a higher treatment success rate, spare the patient unnecessary toxicity and side effects, reduce the cost to patients and insurers of unnecessary or dangerous ineffective medication, and improve patient quality of life, eventually making cancer a managed disease, with follow up assays as appropriate. Physicians can use the reported information to tailor optimal personalized patient therapies instead of the current “trial and error” or “one size fits all” methods used to prescribe chemotherapy under current systems. The inventive methods may establish a system of personalized medicine.

Embodiments of the invention may generate a patient-specific and individualized “wiring diagram” of the cellular circuitry in normal or pathological tissue biopsies. The embodiments may employ biomarkers, which are biochemical characteristics that can be used to measure the progress of a disease or the effects of treatment. The invention may be embodied as a system of molecular medicine, involving the study of and reporting on pathways, which are networks of interacting proteins used to carry out biological functions such as metabolism and signal transduction. Thus, an embodiment of the invention may be a theranostic system that employs biomarkers to guide diagnosis and treatment by physicians, in selecting which patients will respond to what drug.

### Data Processing System

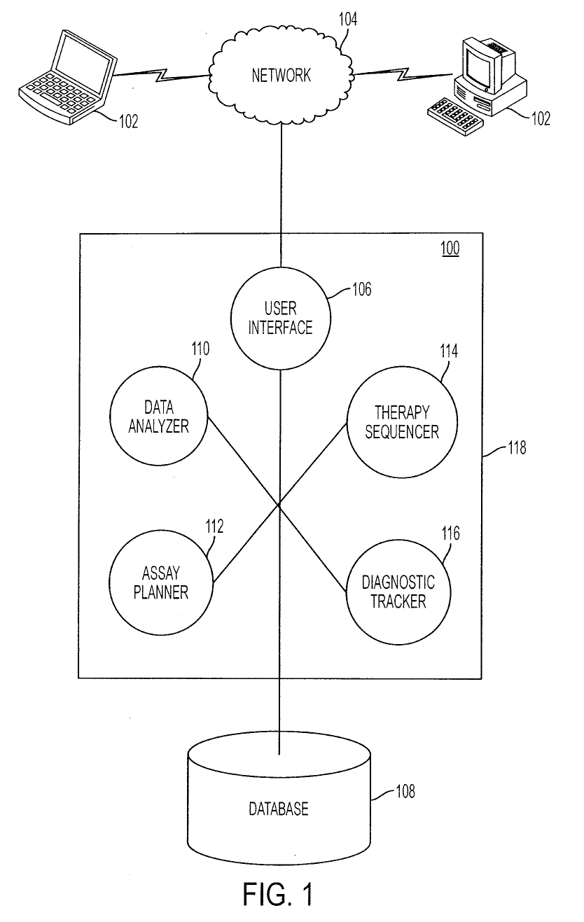

Referring now to the drawings, FIG. 1 depicts a diagram of a system 100 for manipulating theranostic assays in accordance with embodiments of the present invention.

System 100 may be adapted to be accessed by physicians, medical professionals, and/or their assistants using a stand-alone computer (not shown), or one or more of a plurality of networked computers 102 acting as clients. Such clients 102, in turn, may include one or more conventional personal computers and workstations, operating either as a “fat” client or a “thin” client. It should be understood, nevertheless, that other clients 102, such as Web-enabled hand-held devices (e.g., the Palm V™ organizer manufactured by Palm, Inc., Santa Clara, Calif. U.S.A., Windows CE devices, and “smart” phones), which use the wireless access protocol (i.e., WAP), and Internet appliances fall within the spirit and scope of the present invention.

Clients 102 of the above types may access system 100 by way of a network 104. Network 104 may include a number of computers and associated devices that are connected by communication facilities. A network may involve permanent connections such as cables, or temporary connections such as those made through telephone or other communication links. Examples of a network include: an internet, such as the Internet; an intranet; a local area network (LAN); a wide area network (WAN); and a combination of networks, such as an internet and an intranet. By use of the term “network”, it should be understood that the foregoing is not intended to limit the present invention to any particular wireline or wireless network, such as local area networks (i.e., LANs), metropolitan area networks (i.e., MANs), or wide area networks (i.e., WANs). Network 104 may include the Internet (also known as the “World Wide Web”), but it may similarly include intranets, extranets, and virtual private networks (i.e., VPNs) and the like.

In accordance with an embodiment of the invention, system 100 may include a user interface 106, a database 108, a content manager 110, an assay planner 112, a therapy sequencer 114, and a diagnostic tracker 116. Collectively, user interface 106, data analyzer 110, assay planner 112, therapy sequencer 114, and diagnostic tracker 116 may comprise a theranostics application 118.

User interface 106 may be used to interact with system 118, including viewing data and data comparisons graphically. User interface 106 may permit a user to specify, for example, which assay to perform, which biomarkers to collect data for, which data display to use, etc.

Database 108 may include data collected from patients. Database 108 may include aggregated or statistically processed data. The data may be collected from healthy patients and/or from diseased patients. The data may be classified according to disease type. Database 108 may also include data correlating drugs to the biomarkers that the drugs may target.

Data analyzer 110 may analyze patient data and/or data from the database. Data analyzer 110 may compare a patient sample to statistically processed data from database 108 to assist in diagnosis and prognosis of a patient disease. For example, data analyzer 110 may compare a protein profile from a patient with breast cancer to statistically processed data from other breast cancer patients to determine which type of breast cancer the patient has, which cellular pathways may be involved in the patient's cancer, and may suggest one or more drug targets and/or drugs to treat the cancer.

Data analyzer 110 may include a pathway tracker that identifies what signaling pathway is activated or aberrant, and what therapies may be appropriate. Data analyzer 110 may include an assay tracker (not shown) that acquires data about the biomarker assays being conducted, and a report controller (not shown) that selects the appropriate reporting format for the selected assays and data acquired.

Assay planner 112 may determine which panel of target protein biomarkers to assay for a particular patient sample. Assay planner 112 may base the determination on, for example, the source of the patient sample, e.g., breast tissue, liver tissue, etc.

Therapy sequencer 114 may suggest and sequence the course of treatment. Such treatment suggestion could be a single therapeutic agent, or a combination of agents, selected by the specific protein profile match obtained. Therapy sequencer 114 may refer to the data analyzer 110, database 108, and other components of the theranostics system to identify drugs for treating a specific set of target proteins.

Diagnostic tracker 116 may track progress of a patient within the course of treatment.

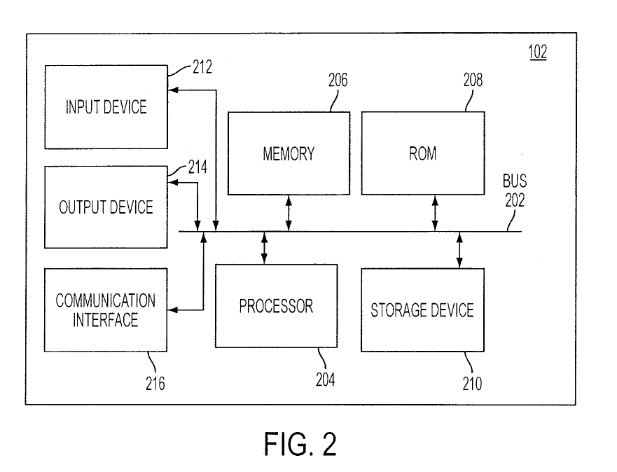

FIG. 2 depicts an exemplary block diagram of a computer 102 that may be configured to execute the theranostics application 118 illustrated in FIG. 1. Computer 102 may include one or more components that may include a bus 202, a processor 204, a memory 206, a read only memory (ROM) 208, a storage device 210, an input device 212, an output device 214, and a communication interface 216.

Bus 202 may include one or more interconnects that permit communication among the components of computer 102, such as processor 204, memory 206, ROM 208, storage device 210, input device 212, output device 214, and communication interface 216.

Processor 204 may include any type of processor, microprocessor, or processing logic that may interpret and execute instructions (e.g., a field programmable gate array (FPGA)). Processor 204 may comprise a single device (e.g., a single core) and/or a group of devices (e.g., multi-core). The processor 204 may include logic configured to execute computer-executable instructions configured to implement one or more embodiments. The instructions may reside in the memory 206 or ROM 208, and may include instructions associated with the TME 104.

Memory 206 may be a computer-readable medium that may be configured to store instructions configured to implement one or more embodiments. The memory 206 may be a primary storage accessible to the processor 204 and may comprise a random-access memory (RAM) that may include RAM devices, such as Dynamic RAM (DRAM) devices, flash memory devices, Static RAM (SRAM) devices, etc.

ROM 208 may include a non-volatile storage that may store information and computer-executable instructions for processor 204. The computer-executable instructions may include instructions executed by processor 204.

Storage device 210 may be configured to store information and instructions for processor 204. Examples of storage device 210 may include a magnetic disk, optical disk, flash drive, etc. The information and computer-executable instructions and information may be stored on a medium contained in the storage device 210. Examples of media may include a magnetic disk, optical disk, flash memory, etc. Storage device 210 may include a single storage device or multiple storage devices. Moreover, storage device 210 may attach directly to computer 102 and/or may be remote with respect to computer 102 and connected thereto via a network and/or another type of connection, such as a dedicated link or channel.

Input device 212 may include any mechanism or combination of mechanisms that may permit information to be input into computer 102 from, e.g., a user. Input device 212 may include logic configured to receive information for computer 102 from, e.g. a user. Examples of input device 212 may include a keyboard, mouse, touch sensitive display device, microphone, pen-based pointing device, and/or biometric input device, etc.

Output device 214 may include any mechanism or combination of mechanisms that may output information from computer 102. Output device 214 may include logic configured to output information from computer 102. Embodiments of output device 214 may include displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc.

Communication interface 216 may include logic configured to interface computer 102 with network 104 and enable computer 102 to exchange information with other entities connected to network 104. Communication interface 216 may include any transceiver-like mechanism that enables computer 102 to communicate with other devices and/or systems, such as a client, a server, a license manager, a vendor, etc. The communications may occur over a communication medium, such as a data network. Communication interface 216 may include one or more interfaces that are connected to the communication medium. The communication medium may be wired or wireless. Communication interface 216 may be implemented as a built-in network adapter, network interface card (NIC), Personal Computer Memory Card International Association (PCMCIA) network card, card bus network adapter, wireless network adapter, Universal Serial Bus (USB) network adapter, modem or any other device suitable for interfacing computer 102 to any type of network.

It should be noted that embodiments may be implemented using some combination of hardware and/or software. It should be further noted that a computer-readable medium that comprises computer-executable instructions for execution in a processor may be configured to store various embodiments. The computer-readable medium may include volatile memories, non-volatile memories, flash memories, removable discs, non-removable discs and so on. In addition, it should be noted that various electromagnetic signals such as wireless signals, electrical signals carried over a wire, optical signals carried over optical fiber and the like may be encoded to carry computer-executable instructions and/or computer data that embodiments of the invention on e.g., a communication network.

Embodiments may be embodied in many different ways as a software component. For example, it may be a stand-alone software package, or it may be a software package incorporated as a “tool” in a larger software product, such as, for example, a medical diagnostic product. It may be downloadable from a network, for example, a website, as a stand-alone product or as an add-in package for installation in an existing software application. It may also be available as a client-server software application, or as a web-enabled software application.

### Theranostic Analysis

Theranostic analyses according to embodiments of the present invention may be carried out by reverse protein microarray techniques, e.g. as described in Sheehan et al., “Use of Reverse Phase Protein Microarrays and Reference Standard Development for Molecular Network Analysis of Metastatic Ovarian Carcinoma,” Mol. Cell. Proteomics, 2005 (4): 346-355, and Liotta et al., U.S. Pat. No. 6,969,614, “Methods for the isolation and analysis of cellular protein content,” which is incorporated herein by reference. Use of such techniques for pathway mapping is exemplified e.g. in Petricoin et al., “Phosphoprotein Pathway Mapping: Akt/Mammalian Target of Rapamycin Activation Is Negatively Associated with Childhood Rhabdomyosarcoma Survival,” Cancer Research 67(7) (2007) (incorporated herein by reference). Further examples of suitable theranostic assays include those disclosed in: PCT Publication No. WO2007/047754, U.S. Patent Publication No. 2007-0224644A1, PCT Publication No. WO2007/106432, PCT Publication No. WO2007/136822, U.S. Pat. No. 6,969,614, U.S. patent application Ser. No. 10/798,799, PCT Application No. PCT/US2007/002452, PCT Application No. PCT/US2007/022744, and PCT Application No. PCT/US2007/022790, all of which are incorporated herein by reference.

The following markers may be used in the theranostic (including diagnostic) panels according to the invention. These phospho-proteins and whole proteins are considered to be markers because an abnormal level in one or more of them is associated with one or more disease states. For each protein, a normal range can be determined, and then the actual level or activity level for a given tissue can be measured and compared to the normal range. The levels of these phospho-proteins may be determined by conventional methods, e.g. with antibodies commercially available from Cell Signalling, Becton Dickinson, Zymed, Stressgen, BioSource, DAKO, Abeam, LabVision, Promega, Upstate, or Santa Cruz.

The following table includes 169 commercially available antibodies to protein markers, some or all of which may be used as markers for an activated (phosphorylated) state of a drug target protein within a tissue, according to embodiments of the present invention. The antibodies may be tested for specificity and cross-reactivity and an appropriate concentration may be selected for optimal performance in an array assay.

TABLE 1
Antibodies to protein markers

| | MODIFIED |
|---|---|
| ENDPOINT | RESIDUES |
| Acetyl and Phospho- | Lys9/Ser10 |
| Histone H3 | |
| Acetylated-p53 | Lys382 |
| Acetyl-beta-Catenin | Lys49 |
| Acetyl-CBP/p300 | Lys1535/Lys1499 |
| Acetyl-Histone H2A | Lys5 |
| Acetyl-Histone H2B | Lys12 |
| Acetyl-Histone H2B | Lys20 |
| Acetyl-Histone H2B | Lys5 |
| Acetyl-Histone H3 | Lys23 |
| Acetyl-Histone H3 | Lys9 |
| Acetyl-Histone H3 | Lys9/Lys14 |
| Acetyl-Histone H4 | Lys12 |
| Acetyl-Histone H4 | Lys8 |
| Acetyl-NF-kappa-B | Lys310 |
| p65 | |
| Acetyl-p53 | Lys379 |
| Acetyl-Stat3 | Lys685 |
| alpha-Fodrin | cleaved (D1185) |
| Caspase-3 | cleaved (D175) |
| Caspase-6 | cleaved (D162) |
| Caspase-7 | cleaved (D198) |
| Caspase-9 | cleaved (D315) |
| Caspase-9 | cleaved (D330) |
| PARP | cleaved (D214) |
| Methyl-Histone H3 | Arg2 |
| 4E-BP1 | S65 |
| 4E-BP1 | T37/46 |
| 4E-BP1 | T70 |
| Acetyl-CoA | S79 |
| Carboxylase | |
| Ack1 | Y284 |
| Adducin | S662 |
| Akt | S473 |
| Akt | T308 |
| Akt1/PKB alpha | S473 |
| AMPKalpha1 | S485 |
| AMPKBeta1 | S108 |
| A-Raf | S299 |
| Arrestin1 (Beta) | S412 |
| ASK1 | S83 |
| ATF-2 | T69/71 |
| ATF-2 | T71 |
| ATP-Citrate Lyase | S454 |
| ATP-Citrate Lyase | Ser454 |
| Bad | S112 |
| Bad | S136 |
| Bad | S155 |
| Bcl-2 | S70 |
| Bcl-2 | T56 |
| c-Abl | Y245 |
| Catenin (beta) | T41/S45 |
| CENP-A | Ser7 |
| Chk1 | S345 |
| Chk2 | S33/35 |
| c-Kit | Y703 |
| c-Kit | Y719 |
| c-Kit | Y721 |
| Cofilin | S3 |
| cPLA2 | S505 |
| c-Raf | S338 |
| CREB | S133 |
| Crkll | Y221 |
| EGFR | S1046/1047 |
| EGFR | Y1045 |
| EGFR | Y1068 |
| EGFR | Y1148 |
| EGFR | Y1173 |
| EGFR | Y845 |
| EGFR | Y992 |
| elF4E | S209 |
| elF4G | S1108 |
| Elk-1 | S383 |
| eNOS | S113 |
| eNOS | S1177 |
| eNOS/NOS III | S116 |
| ErbB2/HER2 | Y1248 |
| ErbB3/HER3 | Y1289 |
| ERK 1/2 | T202/Y204 |
| Estrogen Rec alpha | S118 |
| Etk | Y40 |
| Ezrin | Y353 |
| Ezrin/Radixin/Moesin | T567/T564/T558 |
| FADD | S194 |
| FAK | Y397 |
| FAK | Y576/577 |
| FKHR | S256 |
| FKHR/FKHRL1 | T24/T32 |
| FLT3 | Y842 |
| Gab1 | Y627 |
| GSK-3alpha | S21 |
| GSK-3alpha/beta | Y279/Y216 |
| GSK-3alpha/beta | S21/9 |
| Histone H3 | S28 |
| Histone H3 | S10 |
| Histone H3 | T11 |
| PLCgamma1 | Y783 |
| Bcr | Y177 |
| HP1-gamma | S83 |
| IGF-1 Rec/Insulin | Y1131/Y1146 |
| Rec | |
| IGF-1R/IR | Y1135/36/Y1150/51 |
| IGF-1R/IR | Y1135/36/Y1150/51 |
| IkappaB-alpha | S32 |
| IkappaB-alpha | S32/36 |
| IRS-1 | S612 |
| Jak1 | Y1022/1023 |
| Jak2 | Y1007/1008 |
| Lck | Y505 |
| LKB1 | S334 |
| LKB1 | S428 |
| MAPK | pTEpY |
| MARCKS | S152/156 |
| MEK1 | S298 |
| MEK1/2 | S217/221 |
| Met | Y1234/1235 |
| MSK1 | S360 |
| mTOR | S2448 |
| mTOR | S2481 |
| NF-kappaB p65 | S536 |
| p27 | T187 |
| p38 MAP Kinase | T180/Y182 |
| p40 phox | T154 |
| p70 S6 Kinase | S371 |
| p70 S6 Kinase | T389 |
| p70 S6 Kinase | T412 |
| p90RSK | S380 |
| PAK1/PAK2 | S199/204/S192/197 |
| Paxillin | Y118 |
| PDGF Receptor alpha | Y754 |
| PDGF Receptor beta | Y716 |
| PDGF Receptor beta | Y751 |
| PDK1 | S241 |
| PKA C | T197 |
| PKC (pan)/betaII | _/S660 |
| PKC alpha | S657 |
| PKC alpha/beta II | T638/641 |
| PKC delta | T505 |
| PKR | T446 |
| PKC theta | T538 |
| PKC zeta/lambda | T410/403 |
| PRAS40 | T246 |
| PTEN | S380 |
| Pyk2 | Y402 |
| Ras-GRF1 | S916 |
| RSK3 | T356/S360 |
| Histone H3 Mitosis | S10 |
| Marker | |
| S6 Ribosomal Protein | S235/236 |
| S6 Ribosomal Protein | S240/244 |
| SAPK/JNK | T183/Y185 |
| SEK1/MKK4 | S80 |
| Shc | Y317 |
| SHIP1 | Y1020 |
| SHP2 | Y542 |
| SHP2 | Y580 |
| Smad2 | S465/467 |
| Src | Y527 |
| Src Family | Y416 |
| Stat1 | Y701 |
| Stat3 | S727 |
| Stat3 | Y705 |
| Stat5 | Y694 |
| Stat6 | Y641 |
| Syk | Y525/526 |
| Tuberin/TSC2 | Y1571 |
| Tyk2 | Y1054/1055 |
| VEGFR 2 | Y1175 |
| VEGFR 2 | Y951 |
| VEGFR 2 | Y996 |
| Zap-70/Syk | Y319/Y352 |
| Zap-70/Syk | Y319/Y352 |

The following table (Table 2) includes 92 antibodies to whole proteins, some or all of which may be used according to embodiments of the present invention, similarly to the phospho-specific antibodies listed in Table 1.

TABLE 2
Antibodies to whole protein markers

14-3-3 zeta, gamma, eta
4E-BP1
Abl SH2 domain
Actin, Beta
Akt
Akt2
Aldehyde Dehydrogenase 1
Annexin I
Annexin II
APC2 Ab-1
Aurora A/AIK
Bad
Bak
Bax
Bcl-xL
Bub3
E-Cadherin
Caspase-3
Caspase-7
Caspase-8
Caspase-9
Catenin (beta)
CD3 epsilon
CD3 zeta
CD45
CD133
CDK2
CDK7
CDK9
c-myc
Cofilin
Cox-2
CREB
Crystallin, alpha/Beta
Cu/Zn Superoxide Dismutase (SOD)
Cyclin A
Cyclin B1
Cyclin D1
Cyclin E
EGFR
EGFR (L858R Mut-Spec)
elF4G
eNOS
ErbB2/HER2
ERK ½
Estrogen Rec alpha
FAK
GFAP
GRB2
GSK-3beta
Heme-Oxygenase-1
HIF-1alpha
Histone H3, Di-Methyl (Lys9)
Histone H3, Di-Methyl (Lys27)
Histone H3, Pan-Methyl (Lys9)
HSP70
HSP90
Ig Light Chain, Kappa
IGF-1 Receptor beta
IkappaB-alpha
IRS-1
Kip1/p27
c-Kit
Lck
LEDGF
MARCKS
MEK1/2
MGMT
mTOR
Musashi
NF-kappaB
p38 MAP Kinase
p70 S6 Kinase
pCTD (RNA PCD1)
PDGF Receptor beta
PI3-Kinase
PKC alpha
PLC-gamma-1
PLK1
PTEN
Ras-GRF1
SAPK/JNK
Smac/Diablo
SGK1
c-Src (SRC 2)
Stat3
Stat5
c-Src (SRC 2)

A panel according to embodiments of the present invention may include some or all of the following analytes, in a tabular form like the following Table 3. In the following, phospho AKT and phospho EGFR are elevated out of normal range but phospho ERK is not.

TABLE 3

| ANALYTE | activity value | Normal Range |
|---|---|---|
| phosphoERK | 3.1 | 1.5-5.0 |
| phospoAKT | 10.5 | 1.0-3.0 |
| phosphoEGFR | 22.5 | 0.5-12.0 |

Normal (reference) ranges may be determined based on published data, retrospective studies of sick patients' tissues, and other information as would be apparent to a person of ordinary skill implementing the methods of the invention. The normal ranges may be selected using statistical tools that provide an appropriate confidence interval so that measured levels that fall outside the normal range can be accepted as being aberrant from a diagnostic perspective, and predictive of therapeutic efficacy of modulators of any analytes that fall outside the normal range.

Table 3 is merely illustrative of a data display. A larger panel may include some or all of the following analytes of Table 4.

TABLE 4

| Analyte | Normal Range | Measured Level |
|---|---|---|
| total erB2 | | |
| phosphorylated erbB2: Tyr1248 | | |
| total EGFR | | |
| phosphorylated EGFR: Tyr1148 | | |
| phosphorylated EGFR Tyr1173 | | |
| phosphorylated EGFR Tyr1068 | | |
| phosphorylated EGFR Tyr992 | | |
| IGF-1R phosphorylated Tyr1131 | | |
| phosphorylated AKT ser 473 | | |
| PTEN: ser380 | | |
| Phospho mTOR | | |
| Phospho 4EBP1 | | |
| Phospho NFkB | | |
| Phospho ERK | | |
| Phospho Gsk3b | | |
| Phospho erbB3 | | |
| Total erbB3 | | |
| Phospho estrogen receptor | | |
| Total estrogen receptor | | |
| Total androgen receptor | | |
| Phopsho androgen receptor | | |
| Phospho STAT1 | | |
| Phospho STAT3 | | |
| Phospho PKCalpha | | |
| Phospho p38 | | |
| Phospho S6 | | |
| Cleaved caspase 3 | | |
| Cleaved caspase 9 | | |
| Phopsho lck | | |
| Phospho zap70 | | |
| Phospho ckit | | |
| Phospho abl | | |
| Phospho PDGFR | | |
| Phospho vegfr | | |
| Total vegfr | | |
| Phopsho CREB | | |
| total erB2 | | |

In an embodiment of the invention, reverse phase protein microarray analysis of phosphorylated/activated protein endpoints may be used. The primary targets may be:

HER2

total erbB2

phosphorylated erbB2: Tyr1248

p95/p185

EGFR

phosphorylated EGFR: Tyr1148, Tyr1173, Tyr1068, Tyr992

IGF-1R phosphorylated Tyr1131

AKT

phosphorylated AKT: ser 473, Thr308

PTEN

phosphorylated PTEN: ser380

Additional erbB1/2 downstream endpoints for phosphorylation specific proteins may include, e.g., GSK3α/β ser21/9, GSK3α/β Y279/Y216, mTOR ser2448, MEK ser217/221, Smad2 ser465/467, ERK T202/Y204, and p70S6 Thr389, CD24/44, PI3K.

To provide for quality control, each protein micro-array may contain antigen controls, cell lysate controls, and a reference lysate. Each patient analyte sample can be normalized to total protein and quantitated in units relative to the reference “printed” on the same array. Each reference and control lysate can be printed in the same dilution series as patient samples and be immunostained at the same time, with identical reagents as the patient samples. For controls, one may use A431, A431+EGF, and BT474 cell lysates as the control lysates (including control for p95). All samples can be printed in duplicate in 4-point dilution curves.

To provide for quality assurance, samples can be processed and analyzed in real time as they are received at a suitable processing facility that meets applicable regulatory standards. Samples may consist of Cytolyte preserved samples. A test set with matched frozen samples can verify the adequacy of specimen preservation. Techniques can be carried out at room temperature. Samples may be obtained by core needle biopsy.

There are many examples of depictions of cellular and molecular pathways that may be used to graphically present expected and measured data for selected protein biomarkers (otherwise referred to as targets or reporter proteins). For example, the Reactome website presents many signaling pathways, as do the commercial websites for Sigma-Aldrich and Cell Signaling Technology. Examples of pathway graphics that may be used include the following (and many more):

Akt/PKB signaling pathways

kinase, phosphatase, and other targets in the Akt and other pathways

p44/42 MAP Kinase (Erk 1/2) signaling pathway

estrogen receptor pathways involving Akt and MAPK

EGF receptor signaling pathway

HER2/ErbB2 signaling pathway.

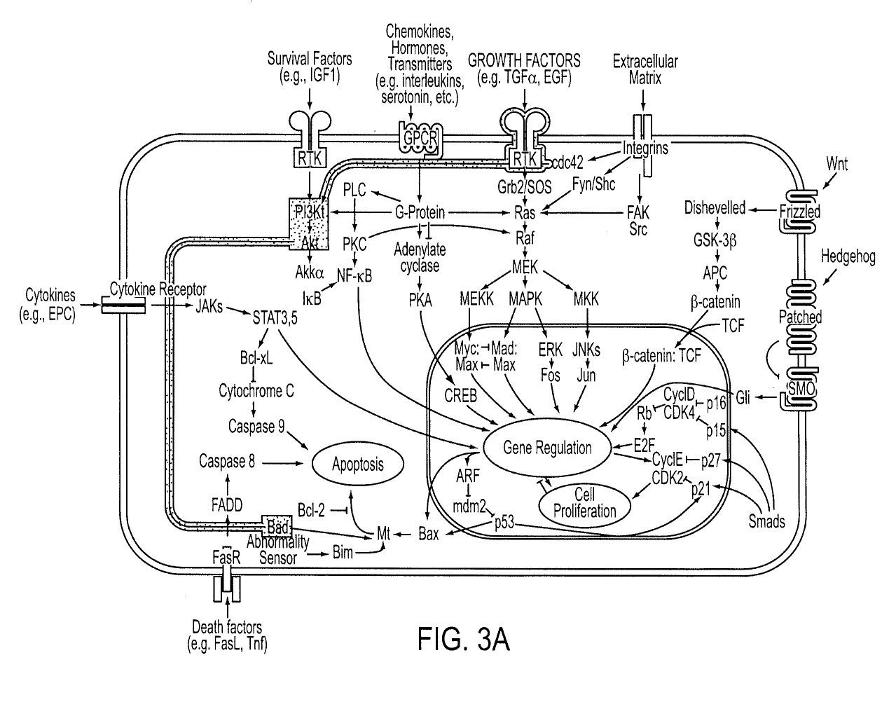

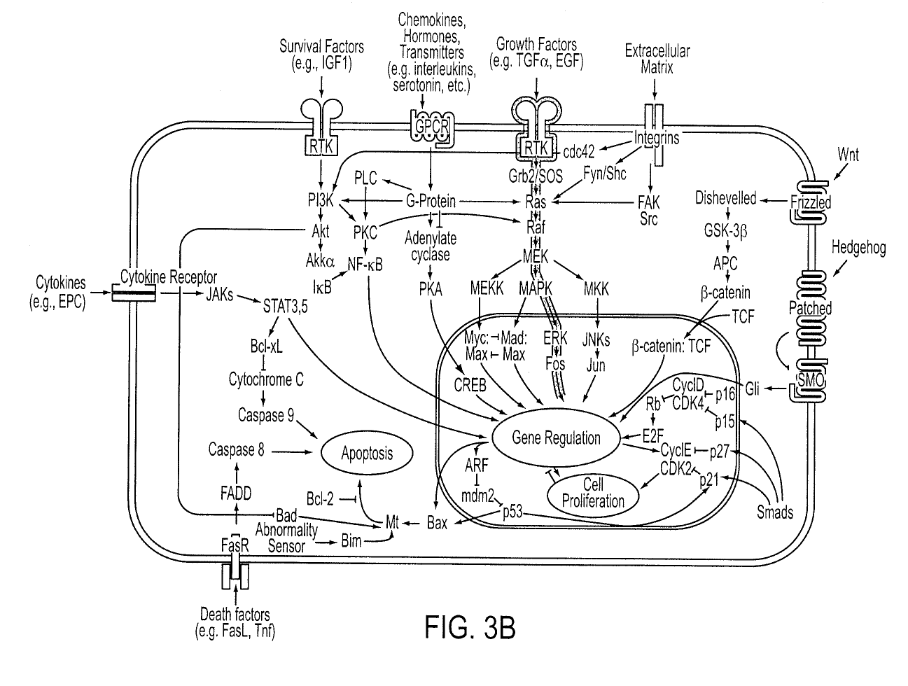

FIG. 3 is adapted from an open access signaling pathway image on Wikipedia, and shows an example of a diagram report for two patients, according to an embodiment of the invention, using signaling pathway highlights. A person of ordinary skill may correlate the protein marker that is used in the reporting panel or display with the protein marker's location in the appropriate cell signaling pathway diagram. Such a diagram may be used to indicate which marker is at an aberrant level, outside the normal range.

In FIG. 3A, an activated Akt pathway is shown as a highlighted pathway for patient A, for whom the measured protein modification levels of proteins in that pathway are outside the reference range. Likewise, in FIG. 3B, a MAPK pathway is shown as a highlighted pathway for patient B.

In FIG. 3B, for patient B, one or more biomarkers in the highlighted pathway B are active, suggesting a different therapeutic target. The different pathways suggest different therapeutic targets. Different therapeutic targets may suggest different drugs. Therefore, patient A and patient B may be prescribed a different drug according to their respective pathways.

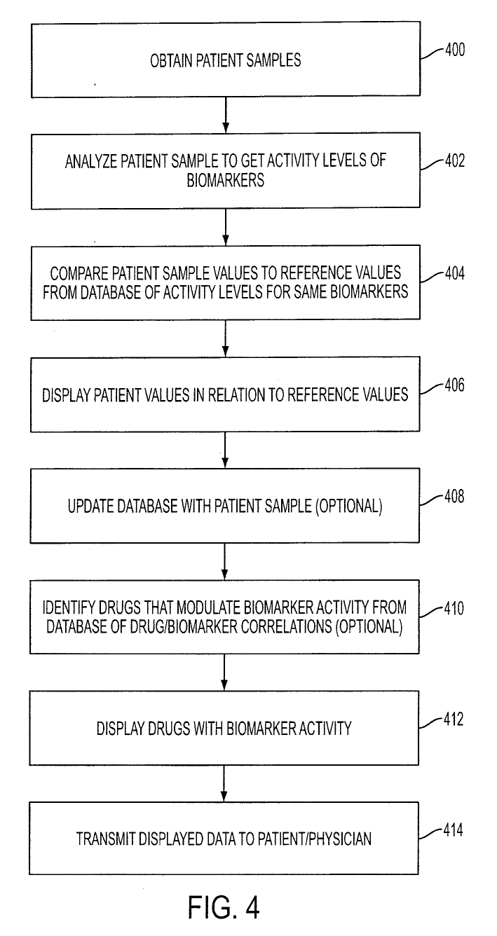

FIG. 4 depicts a flowchart of a technique for manipulating theranostic arrays. In block 400, a patient sample may be obtained. Examples of patient samples may include, for example, cells grown in culture stimulated with ligand and/or drug ex vivo, laser capture microdissected cells, FACS sorted cells, blood cells, touch prep, fine needle aspirant, core biopsy, non-cellular body fluid (e.g. vitreous, urine, nipple fluid aspirate, sweat, tear, saliva, etc.), magnetic bead sorted, etc.

In block 402, the patient sample may be analyzed to determine the activity level of a set of protein biomarkers in the sample. Examples of analysis techniques may include, for example, RPMA, ELISA, suspension bead array (e.g. Luminex), surface plasmon resonance, evanescent wave, cantilever based, nano-sensors, immunofluorescence, immunohistochemistry, etc.

The patient sample may include protein biomarker data for a signaling pathway protein that is modified chemically in a process of post-translational modification. Such modification may include, for example, phosphorylation, sumoylation, myristylation, farnesylation, acetylation, sulfonation, or glycosylation. An activity level for such a protein may correspond to degree of modification which can be measured by techniques known to one of ordinary skill in the art. Such measurements may be immunoassay-based (e.g. ELISA, immunohistochemical, reverse phase array, flow-sorted, suspension bead array), and may be performed with antibodies that are specific against modified forms of the protein analyte of interest, such as sumoylation-specific, acetylation-specific and other antibodies (Doman et al., DNA-dependent acetylation of p53 by the Transcription Coactivator p300*, J. Biol. Chem., Vol. 278, Issue 15, 13431-13441, Apr. 11, 2003; Chen et al., Use of a new polyclonal antibody to study the distribution and glycosylation of the sodium-coupled bicarbonate transporter NCBE in rodent brain, Neuroscience, 2008 Jan. 24; 151(2):374-85, Epub 2007 Oct. 25.)

In block 404, the activity levels from the patient sample may be compared to a set of reference values. The reference values may be determined from a collection of activity level values for samples related to the patient sample. The reference values are said to be related to the patient sample if the patient and reference values share certain disease characteristics. For example, a sample from a patient with metastatic breast cancer may be compared to reference data from other patients with metastatic breast cancer. Other disease characteristics that may be considered to select appropriate reference values may include: a general cell type (e.g. epithelial, stromal, or hematopoetic), cancer, normal, premalignant, etc. Additional characteristics that may be considered in selecting reference values may include, for example, cancer type, grading, staging, pathologic diagnosis, type of metastasis, epidemiological parameters (e.g. menopausal status, age, sex, etc.), pre- or post-treatment, type of treatment, etc.

The reference values may be statistically processed to provide values for comparison. For example, the reference values may provide an average activity level, a standard range of activity level, a standard cell pathway, etc., for a particular biomarker or set of biomarkers. Statistical processing may include, for example, standard power calculations based on assumptions based on distribution of the population data, e.g. normal distribution vs. abnormal distribution using parametric or non-parametric statistics (e.g anova, kruskall wallis, wilcoxon rank sum, students t-test, etc.

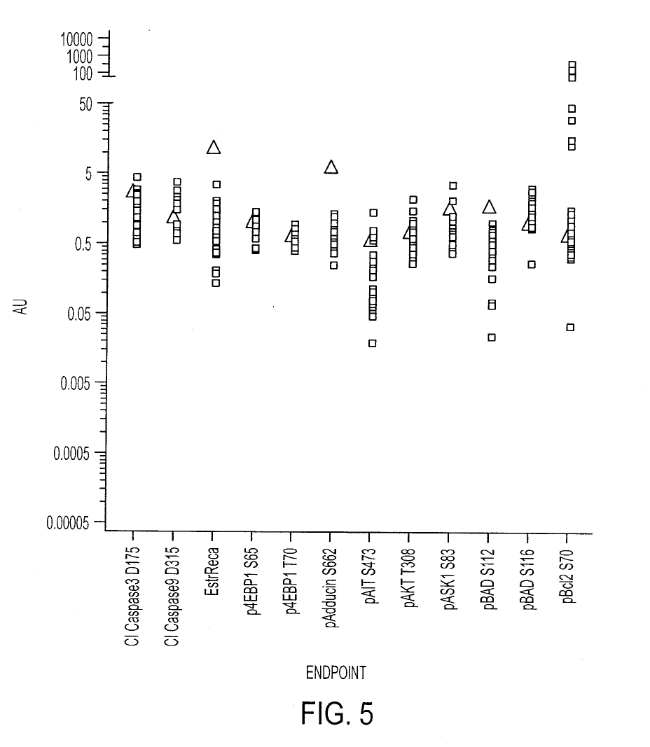

In block 406, the values from the patient sample may be displayed in relation to the reference values. Display may include displaying on a screen, printing, or outputting to another device for storage or further processing. Examples of displays are discussed further below with respect to FIGS. 5-16

In block 408, the patient data may be optionally added to the reference data as another sample. The reference data may be re-processed statistically to include the patient data. Any patient values and the corresponding response rates may be added to the growing and updated reference data, including data about ranges of activation that correlate with response to therapy as more data is collected.

As shown in block 410, the system may optionally identify drugs that modulate the activity of some or all of the selected biomarkers, in reference to a database of drug/biomarker correlations. The database of drug/biomarker correlations may be contained in database 108, or be a separate database accessible to the system.

In block 412, the names of drugs correlated with activity of a particular biomarker may be displayed.

In block 414, the displayed drug information can be transmitted to the patient via the physician, from the testing laboratory, by any means evident to persons of ordinary skill.

These steps may be done in other orders or simultaneously. For example, patient data can be obtained and compiled to prepare and update a database with patient sample data, before or separately from providing a comparative analysis for any particular patient. Also, the identification of correlated drugs may be completed before or simultaneously with analyzing and displaying patient samples, e.g. along with the reference values.

Examples of Data Displays

FIG. 5 shows an example of a scatterplot of set of biomarkers along the X axis, with boxes indicating normalized activity levels collected from a set of patients, and values for the patient being analyzed shown in triangles. The activity level values may be shown in arbitrary units and in a logarithmic scale for compact viewing. In the scatterplot of FIG. 5, the cumulative data (i.e., unfilled boxes) is presented with the unconnected line plot exhibiting the patient sample (i.e., unfilled triangles). The data points are unobstructed by summation devices that place a rectangle around points of most concentration, but visually, the data may be very busy due to the large number of cumulative data points. This aspect may also make the trend for the unconnected patient sample line somewhat hard to follow. Other approaches can be used for showing the data in a clear way that can be interpreted at a glance.

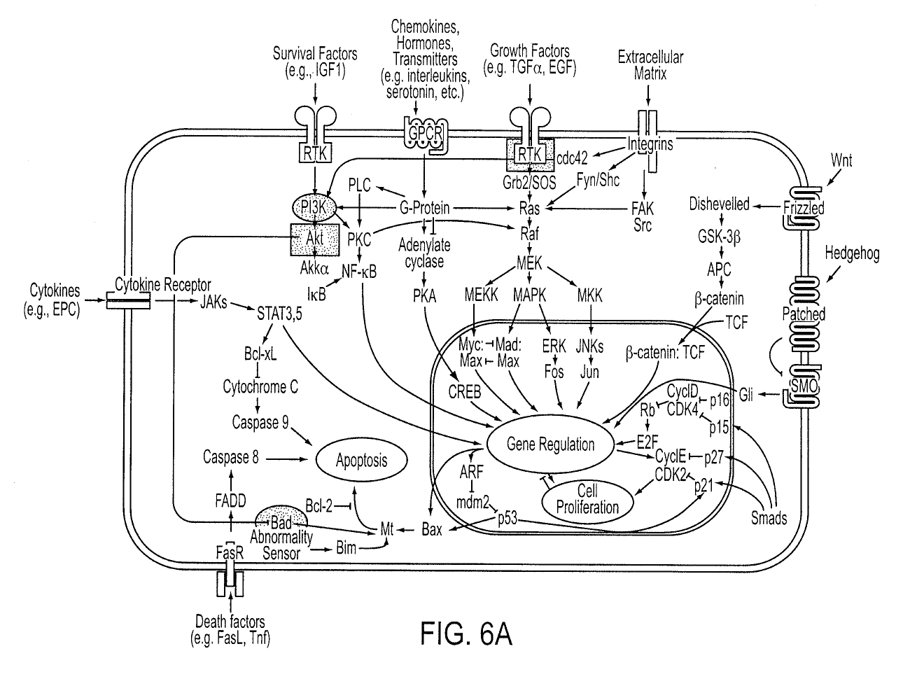

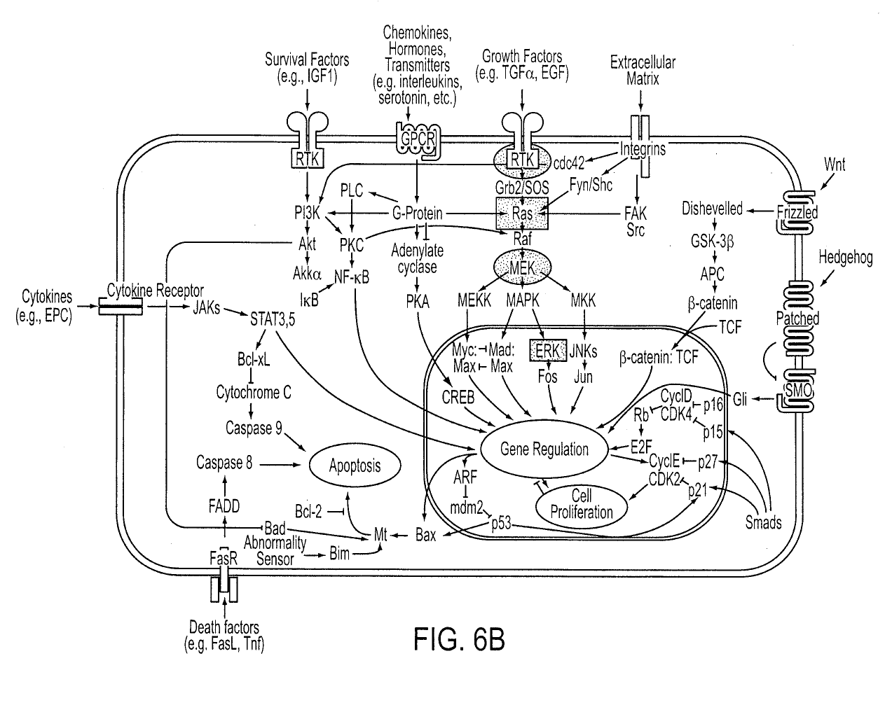

FIG. 6 shows a signaling network diagram where the biomarkers having notable activity are shown directly in place in the relevant signaling pathways, so that patterns and associations between them may be readily apparent. For example, FIG. 6A shows activation of an Atk signaling pathway, and FIG. 6B shows activation of a MAPK signaling pathway. In FIG. 6A, RTK and Akt are shown with shaded boxes, to show activity in the top decile of patients, and P13K and Bad are shown with shaded ovals, to show an activity in the top quarter of samples. This pattern indicates activation of the Akt pathway in patient A. In FIG. 6B, Ras and Erk are shown with shaded boxes (top decile), and RTK and MEK are shown with shaded ovals (top quarter), together indicating activation of the MAPK/Erk pathway for patient B.

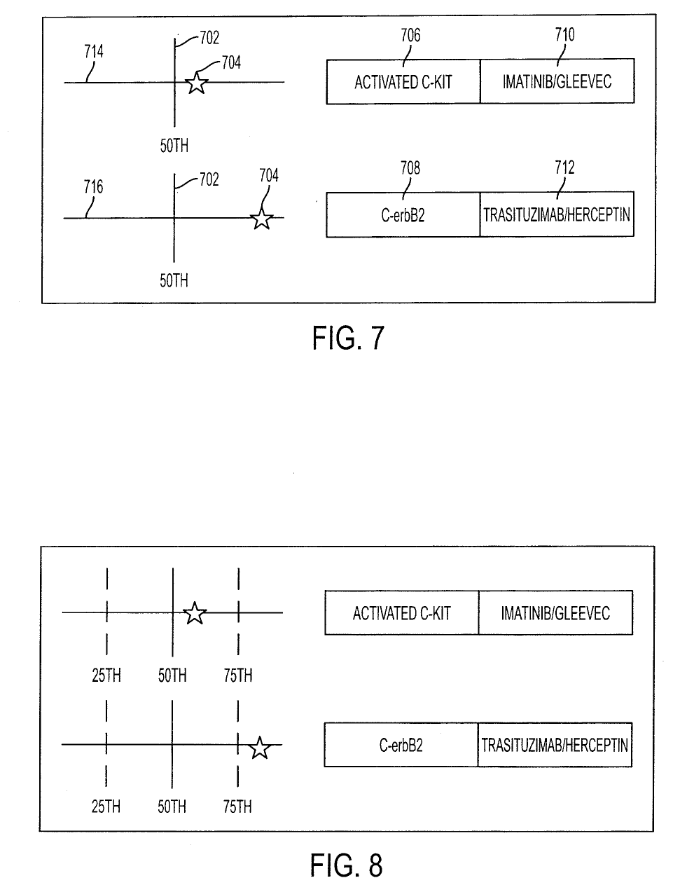

FIG. 7 shows a variant of a Drug Target Activity Report, with the median point shown as a hash mark 702 on a linear scale, and the patient data shown as a star 704. For the two lines shown, the drug targets are Activated C-Kit 706 and C-erbB2 708, and the corresponding FDA approved drugs are Imatinib/Gleevec 710 and Trasituzimab/Herceptin 712. The patient data for the top line 714 is near the median, while the patient data for the bottom line 716 is far above the median.

FIG. 8 shows another variant of a Drug Target Activity Report, similar to that in FIG. 7, with the 25^{th}, 50^{th}, and 75^{th} percentiles point shown as hash marks on a linear scale, and the patient data shown as a star. For the top line, the patient data is close to the 50^{th} percentile, and for the bottom line the patient data is far beyond the 75^{th} percentile (in the top decile).

An “abnormal” level, or a level “outside the normal range” typically refers to a level in excess of a normal or reference range. In some examples, the “abnormal” level may be below the normal (reference) range.

The normal range can be determined by one or more methods. For example, the values for a particular marker in cells of a cell line can be measured (a) in their unstimulated condition, (b) after introducing an agent that models a pathological condition such as a mitogen, that modulates the modification of a target protein, and (c) adding an inhibitor of the pathogenic agent. The cell line may be HeLa cells, the pathogenic agent may be EGF, and the inhibitor may be an EGF inhibitor. The normal value would be determined from the range observed in (a) and/or (c) above, and would be distinct from the range observed in (b). Alternatively or in addition, retrospective data may be obtained from sick and healthy patient samples, where the values of a marker in the healthy patient samples determine the normal range, as distinguished from the range of values in the sick patient samples. The ranges may be determined in a manner that would be apparent to a person of ordinary skill, e.g., using statistical tools.

In another embodiment, the invention involves drug screening. A cell line or tissue in a pathological condition (such as in situation (b), above) can be used as a control, and various putative inhibitors can be administered, to determine if any of them restore a normal level of activity for the given marker, indicating that the putative inhibitor is potentially therapeutic, that is, a lead compound for further drug screening tests. The effect of a putative inhibitor can be compared to the effect of a known inhibitor.

The analytes may be selected from the lists provided herein above, or a subset thereof, or other analytes identified by a practitioner according to the invention. The analytes may be proteins, or post-translationally modified protein isoforms, e.g., phosphorylated, cleaved, or glycosylated proteins, provided that the isoform can be recognized specifically by a suitable antibody to that isoform. Phosphorylated proteins are advantageous markers because their quantitative level indicates an activity level for that protein. The activity values for the selected analytes or markers are strongly predictive of particular disease conditions, with much higher specificity than, say, the components measured in a standard blood test.

Some embodiments of the invention, as discussed above, may be embodied in the form of software instructions on a machine-readable medium. Such embodiments are illustrated in FIGS. 1 and 2.

According to the invention, data may be acquired for several or many different markers. Using reverse protein micro-arrays, a sample containing only about a thousand cells may be used to measure the activity of hundreds of cell signaling proteins, or a smaller more select group of proteins depending on the theranostic need. Once data is acquired, data for all or less than all of the markers may be reported. Reporting of all or less than all of the acquired data according to the invention may be in a convenient format readily used by physicians to determine therapy for patients. For example, this may be a table listing levels for all tested protein markers, one by one, with normal levels. Alternatively, the report may include only those markers for which the activity level is abnormal, providing a simpler reporting format. Or the report may include those markers associated with a particular cancer type, e.g. breast, lung, prostate, colorectal, and/or ovarian cancers and/or leukemia, multiple myeloma, and rhabdomyosarcoma.

The selection of markers (either for the overall biological assay that is conducted, or for data gathering, analysis and reporting of resulting data) may involve selecting appropriate signaling pathways, and then selecting those markers that are representative or indicative of an aberration in that particular pathway. That is, if more than one marker could be measured as an indicator for hyperactivation of a particular pathway, there is redundancy, and not all of the redundant markers needs to be tested and/or reported. Such selection may speed analysis, reduce cost, and improve the user interface. The number of markers tested at any given time may be at least or no greater than 3, 5, 10, 15, 25, 30, 50, 75, 100, 150, 200, 250, or 300.

The report may further include information about approved pharmaceutical compounds that are known to impact the particular molecular signaling pathway with abnormal activity in the particular patient. For example, aberrant activity levels for the following analytes are correlated with therapy using the following commercially available drugs shown in Table 5:

TABLE 5

| Example Analyte | Specific Therapy |
|---|---|
| c-erbB2 | HERCEPTIN |
| c-erb1 | TARCEVA |
| c-erb2 and cerbB1 | LAPATINIB |
| c-kit | GLEEVEC |
| VEGFr | AVASTIN |

These drugs may be tested for efficacy in restoring normal activity levels for other signaling pathway markers, and other drugs may likewise be tested in comparison to the approved drugs, as shown in Table 6.

TABLE 6

| | Baseline Population Distribution in Breast | Patient | |
|---|---|---|---|
| Analyte | Cancer Patient | Value | Drug |
| Phospho | 0-100 RU/cell [bottom quartile (LOW)] | | Gleevec |
| c-kit | | | |
| | 101-200 RU/cell [second quartile | | |
| | (Medium-Low)] | | |
| | 201-300 RU/cell [third quartile (Medium- | 358 | |
| | High)] | | |
| | >301 RU/cell [top quartile (High] | | |

An example of a data report according to the invention is provided below in Table 7.

TABLE 7
Reverse Phase Protein Microarray Signal Pathway Profile Report

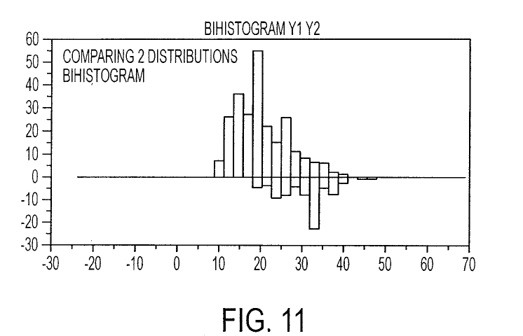

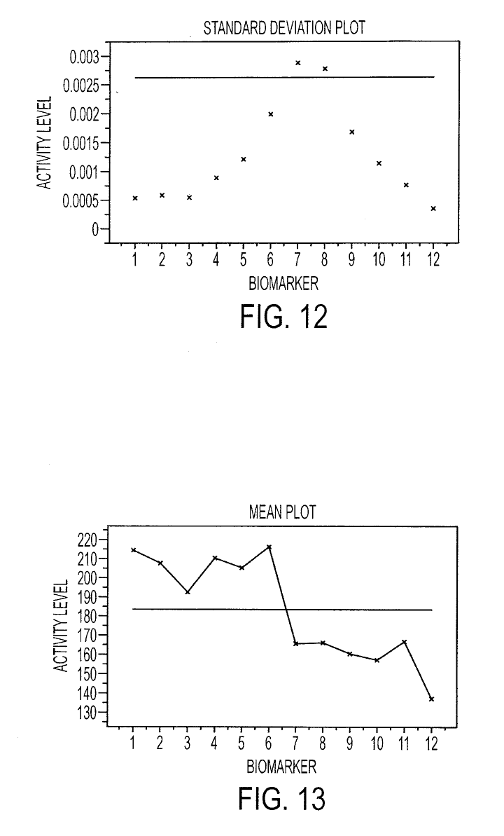

Patient: John Doe
Physician: Dr. Physician
Specimen received: Core Needle Biopsy X 2
Gross Description
Pathology specimens:
Specimen 1: Touch prep diagnosis on specimen B: metastatic carcinoma
Tissue: Liver
Pathologic Diagnosis:
Specimen 1: Liver with 70% replacement by metastatic carcinoma consistent with colorectal
cancer primary
Method
The metastasis region of Specimen 1 was analyzed using reverse phase protein microarray
technology in the CAP/CLIA accredited laboratory located at
Theranostics Health, LLC
15010 Broschart Road
Rockville, MD 20850
Tel: 301-251-4443
The technology has been the subject of nearly 50 peer reviewed publications (sf Gullman et al,
Oncogene, 2007; Petricoin et al, Cancer Research, 2007). The platform is a highly sensitive
protein microarray capable of analysis of protein expression from small biopsy samples that
produces semi-quantitative and quantitative data similar to immunoassay results. The method
produces data that that reveals activation of specific signaling proteins based on their
phosphorylation. Phosphorylation is the critical measurement of pathway activation, thus an
elevated measurement indicates increased activation of the endpoint/pathway.
For this report, the total number of phosphoprotein endpoints quantified was seventy-one (see
attached plot).
Results
For each individual endpoint this patient's profile was ranked by comparison with a control
population of 35 liver metastasis from patients with colon cancer
1. Specimen 1 signal protein analysis of the metastatic region.
1.1 Results for all analyte endpoints are depicted in scattergraph. (FIG. 11). The vertical axis is a
log scale of the normalized analyte intensity value. The horizontal axis is the list of all analytes.
This patient's result for each endpoint is shown as a yellow triangle which can be ranked within
the distribution of control population values (red box) for each analyte.
1.2 Ranking for analysis of the metastatic region of specimen B in a signaling network diagram
(FIG. 12)
The following phosphorylated analytes are ranked in the top 10% of the liver metastasis patient
population values. Phosphorylation is the critical measurement of pathway activation, thus an
elevated measurement indicates increased activation of the endpoint/pathway.

Patient value is within top 10% of all patients analyzed (Phosphorylation site in parenthesis)

| | |
|---|---|
| Drug Target Measured | FDA Approved Therapeutic |

Phosphorylated analytes with an intensity exceeding 90% of control population values.

| | |
|---|---|
| pmTOR (S2448) | Tirisolimus |
| p4EBP1 (T70) | Tirisolimus |
| pFKHRL (S256) | Tirisolimus |
| pEIF4G (S1108) | Tirisolimus |
| pAbl (Y245) | Imatinib |
| pc-kit (Y703) | Imatinib |
| pPDGFR (Y751) | Imatinib |

Phosphorylated Analytes with an intensity exceeding 75-90% of control population values.

| | |
|---|---|
| c-erbB2 | Herceptin/trastuzimab |
| c-erbB1 | gefetinib |

END OF REPORT

### Alternative Display Approaches

Several alternative approaches may be used to display the patient data in comparison to a relevant patient population data set, in addition to the signaling pathway images and scatterplot described above. A person of ordinary skill may select among them depending on the needs and circumstances. Generally, reference values are displayed in arbitrary units, may be normalized, and may be showed in a logarithmic scale to accentuate differences.

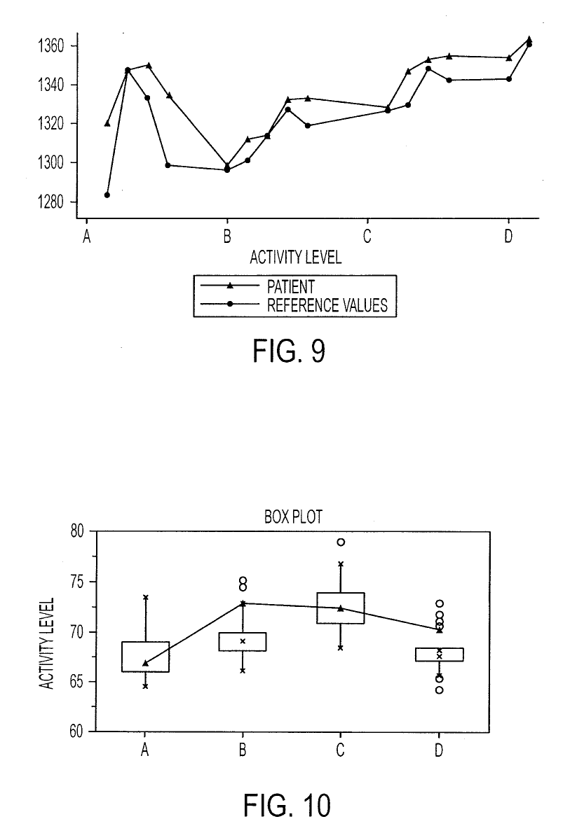

FIG. 9 shows a method of display in which the patient sample values are connected via a line graph plot. This may simplify the graph and help the viewer more easily interpret the data at-a-glance and, for instance, quickly see how many points are above or below the reference value data plot line.

Another way to present cumulative data is with a box plot, as shown in FIG. 10. This is also known as a box-and-whisker plot. The resulting bar presentation of cumulative data provides a simplified version of a scatter plot while continuing to present outliers relative to the patient sample. For example, the triangle point-connected patient line may be graphed on top and separately from the cumulative data box plot. Although adding bars via a box plot may decrease the amount of detail, it permits presentation of a great amount of information in a concise, easy-to-interpret way without sacrificing key data trends. This is made possible by the so-called whiskers of the plot that delineate the range of data.

FIG. 11 shows a bihistogram graph. In this case, one half of the bihistogram may represent the average among all samples (with error bars to indicate sample range) and the other half-histogram may represent the connected line plot of the patient sample. The patient sample may be represented by the top bars. The bars make the data easier to interpret but even with the error bars, less detail of the cumulative data is presented overall.

FIG. 12 shows a standard deviation figure. FIG. 13 shows a mean plot display. These approaches may provide a single reference point for the cumulative data compared to the patient sample data but would show less information than the original plot because range and outliers are not displayed.

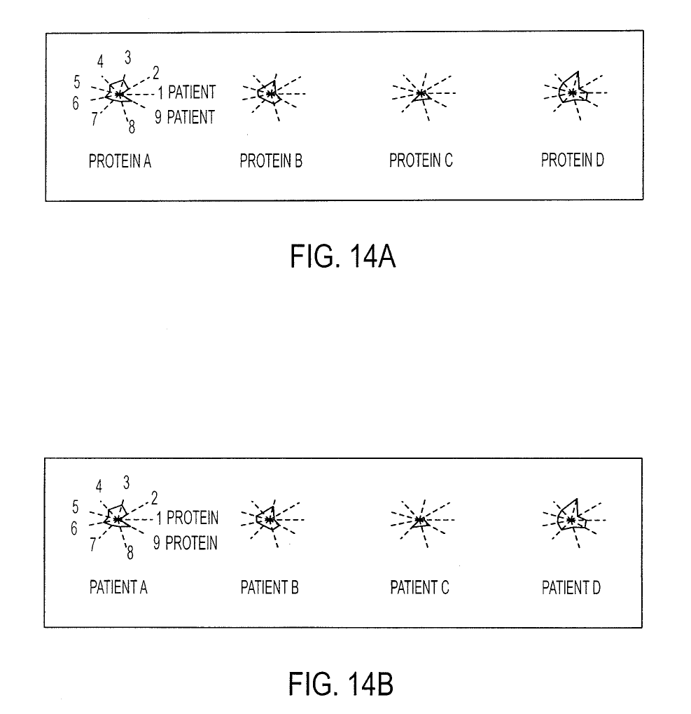

A star plot is shown in FIG. 14. A star plot may indicate how all the specified proteins in the patient sample relate to the cumulative picture of all the other patients when viewed one by one. For example, in FIG. 14A, nine patient samples may include four proteins, A, B, C, D. The patients' activity levels related to each protein may be displayed in a star format. In FIG. 14B, the protein modification levels for nine separate proteins are shown for four different patients. This approach displays much information in a highly detailed view but would require many lines within each star, so is appropriate when showing a smaller number of samples and patients at one time.

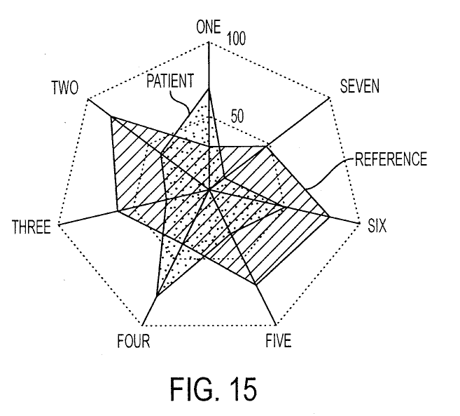

One could present patient data using a radar plot, as shown in FIG. 15. FIG. 15 shows a patient's protein modification levels for seven different proteins related to the statistically processed reference values for the same seven proteins.

Other forms of display will be readily apparent to a person of ordinary skill. These displays may be presented conveniently in digital images, html, ASCII data format, or otherwise, for example on computer screens or printed reports.

Embodiments of the invention has been described in detail with respect to various embodiments, and it will now be apparent from the foregoing to those skilled in the art that changes and modifications may be made without departing from the invention in its broader aspects. The invention, therefore, as defined in the appended claims, is intended to cover all such changes and modifications as fall within the true spirit of the invention.

What is claimed is:

1. A computer-implemented method of manipulating theranostic assays, comprising:
analyzing a sample of cells to obtain a series of sample quantitative values of protein modification levels for a set of target proteins, the set being sufficient to define one or more signaling pathways;
comparing the sample quantitative values to reference quantitative values of protein modification levels for the same set of target proteins, wherein the reference quantitative values are statistically processed from a plurality of comparable samples; and
displaying the sample quantitative values in relation to the reference quantitative values.

2. The method according to claim 1, wherein at least one target protein is a signaling pathway protein that is modified chemically in a process of post-translational modification including at least one of phosphorylation, sumolyation, myristylation, farnyslation, acetylation, sulfonation, or glycosylation, and the quantitative value is a measurement of the level of modification.

3. The method according to claim 1, wherein the quantitative value is a measure of target protein phosphorylation.

4. The method of claim 1, wherein the target protein modification level indicates a likelihood of susceptibility of the cells to a drug that is a modulator for the target protein modification, wherein the method further comprises displaying the name of the drug in conjunction with the protein modulated by the drug.

5. The method according to claim 1, further comprising:
obtaining the reference quantitative values from a statistically significant sample size of patient protein samples;
aggregating reference quantitative values from the patient protein samples; and
statistically processing the aggregated data.

6. The method according to claim 5, wherein statistically processing includes normalizing the aggregated data.

7. The method according to claim 5, further comprising:
updating the reference quantitative values with the sample quantitative values; and
storing the updated reference quantitative values.

8. The method according to claim 1, wherein displaying comprises displaying the set of protein modification levels in a tabular form.

9. The method according to claim 1, wherein displaying comprises displaying said series of protein modification levels in a diagrammatic form.

10. The method according to claim 9, wherein said diagrammatic form comprises at least one of:
one or more diagrams, each of which represent one or more of a plurality of target proteins and/or signaling pathways, selected from
a highlighted signaling pathway diagram;
a scatter plot;
a box plot;
a bihistogram;
a block plot;
a standard deviation plot;
a star plot; or
a radar plot.

11. The method according to claim 1, wherein a quantitative value comprises the level of phosphorylation, and wherein the reference value comprises a level of that phosphorylation in a population of reference samples.

12. The method according to claim 11, where the reference value comprises a level of phosphorylation in a cell line stimulated with a ligand or a phosphatase inhibitor.

13. The method according to claim 11, where the reference value comprises a level of phosphorylation in a purified sample of the analyte of known concentration.

14. The method according to claim 1, further comprising:
identifying a sample quantitative value for a target protein modification level that falls outside of a reference standard range; and
identifying a drug correlated with the target protein based on the identified sample quantitative value.

15. The method according to claim 1, wherein displaying comprises graphically emphasizing a sample value that falls outside of a reference standard range.

16. A computer-readable medium containing software code that, when executed by a processor, performs the method according to claim 1.

17. The method according to claim 1, wherein the cells are from a subject or cell culture.

18. The method according to claim 1, wherein the set of selected target proteins is sufficient to distinguish an activated signaling pathway from a non-activated signaling pathway, and the activated signaling pathway is associated with a disease subcategory.

19. The method according to claim 18, wherein the disease subcategory is selected from the group consisting of: (a) human cancer without regard to type of cancer; (b) diabetes; (c) cardiovascular disease; (d) inflammation; (e) infectious disease; (f) ocular diseases; and (g) neurodegenerative diseases.

20. The method according to claim 17, comprising:
measuring the values for a target protein in cells of a cell line in their unstimulated condition;
measuring the values for the target protein in cells of the cell line after introducing an agent that modulates protein modification in a signaling pathway associated with a pathological condition; and
measuring the values for the target protein modification in cells of the cell line after adding a candidate inhibitor of the modulator.

21. The method according to claim 20, wherein said cell line comprises HeLa cells.

22. The method according to claim 20, wherein said modulator comprises EGF.

23. The method according to claim 20, wherein said inhibitor comprises an EGF inhibitor.

24. The method according to claim 20, wherein said reference standard range is one of determined or selected from the range observed in said measuring steps.

25. The method according to claim 17, wherein the protein modification comprises one or more post-translationally modified protein isoforms.

26. The method according to claim 25, wherein said post-translationally modified protein isoforms are selected from the group consisting of phosphorylated, cleaved, and glycosylated proteins, and the isoform can be recognized specifically by a suitable antibody to that isoform.

27. A theranostics system, comprising:
a computer network, including server means and a plurality of clients in communication with said server means over said network;
means for operating said server means and said plurality of clients, said operating means supporting a run-time environment for a theranostics application on said network;
graphical user interface means adapted to be displayed on said plurality of clients;
a database storing a plurality of files with a plurality of different file formats;
a plurality of collaborative modules, each of which is adapted to be run over said network, said collaborative modules including:
an assay planner to determine which target proteins to assay; and
a data analyzer to analyze a sample of cells to obtain sample quantitative values for a series of target protein modification levels reflected in a set of a plurality of target proteins in the sample and to compare the sample quantitative values to reference quantitative values.

28. The theranostics system of claim 27, further comprising:
a therapy sequencer to sequence a course of treatment; and
a diagnostic tracker to track progress of said patient within said course of treatment.

29. A method of operating a theranostics system, comprising:
obtaining patient data from a client computer;
analyzing the patient data on a server to obtain sample quantitative values for a series of target protein modification levels reflected in a set of a plurality of target proteins in the sample;
comparing the sample quantitative values to reference quantitative values for the same series of target proteins wherein the reference quantitative values are statistically processed from a plurality of comparable samples; and
displaying the sample quantitative values in relation to the reference quantitative values on the client computer.

30. The method of claim 29, further comprising:
conducting an assay on a patient sample to produce the patient data on the client computer prior to the obtaining.

31. A method of characterizing a disease in a subject, comprising:
selecting a set of target proteins of a plurality of cell signaling pathways, wherein the signaling pathways involve modifications to the target proteins,
obtaining a set of quantitative values for a reference range of modification levels for each of the target proteins in the set, wherein the level in one or more of the target proteins indicates a relative state of activation in a signaling pathway in the cell associated with a disease as compared to modifications in target proteins within the reference population of patient values,
measuring modification levels for the set of target proteins in cells of a patient, determining, for each target protein in the set, whether the modification level is high or low compared to the reference population of patients, and
displaying the modification levels of the set of target proteins in the sample and the reference range of modification levels for the target proteins.

32. The method of claim 31, further comprising displaying the signaling pathway or pathways of the target proteins along with their relative modification levels.

33. The method of claim 31, further comprising displaying the identity of one or more drugs that modulate modification levels of the target protein.

34. The method of claim 31, further comprising displaying one or more drugs that modulate activity of the signaling pathway of the target protein(s) with relatively increased modification levels.
